(19) United States
(12) Patent Application Publication
(10) Pub. No.: US 2004/0219593 A1
(43) Pub. Date: Nov. 4, 2004

(54) Biochip and method of designing probes

(75) Inventors: Yasuyuki Nozaki, Kanagawa (JP); Shingo Ueno, Kanagawa (JP); Ryo Nakashige, Kanagawa (JP); Toshiko Matsumoto, Kanagawa (JP); Takuro Tamura, Kanagawa (JP)

Correspondence Address: REED SMITH LLP, Suite 1400, 3110 Fairview Park Drive, Falls Church, VA, 22042, US

(73) Assignee: Hitachi Software Engineering Co., Ltd.

(21) Appl. No.: 10/857,909

(22) Filed: Jun. 2, 2004

Related U.S. Application Data
(63) Continuation of application No. 10/076,932, filed on Feb. 15, 2002.

(30) Foreign Application Priority Data
Mar. 29, 2001 (JP) 2001-96978
May 11, 2001 (JP) 2001-142170

(51) Int. Cl. C12P 21/06; C12Q 1/68

(52) U.S. Cl. 435/6

(57) ABSTRACT

A method of conducting accurate identification of biological species with a biochip, and a method of effectuating identification of biological species at a level higher than species are provided. Selection of specific probes for multiple biological species is also facilitated. A plurality of partial sequences A, A′; B, B′; and so on, which are specific to respective targets, are selected as probes. in a manner that the partial sequences do not overlap one another. In addition, DNA regions I, J, K and L, which are common to some targets, respectively, are also selected as probes. Alternatively, if there is a common base sequence at leaves below a certain node based on a dendrogram of targets, such a base sequence is designed as a probe unique to the node. By using a set of probes including the probes unique to the targets and the probes unique to leaves, identification of biological species can be performed accurately, and identification of biological species at a level higher than species is also effectuated. In a case where the base sequences corresponding to leaves are identical or similar to each other, such base sequences can be used as probes if sequences corresponding to nodes are different. Therefore, selection of specific probes among multiple biological species can be facilitated.

### PRIORITY INFORMATION

This application claims priority to Japanese Application Serial No. 2001-96978, filed Mar. 29, 2001, and to Japanese Application Serial No. 2001-142170, filed Mar. 11, 2001.

### BACKGROUND OF THE INVENTION

1. Field of the Invention

The present invention relates to a biochip for identifying a plurality of biopolymers such as DNA contained in a sample, and to a method of designing probes to be spotted on the biochip.

2. Prior Art

Functions and structures of genes are gradually coming out by virtue of development in gene analytic technologies in recent years. Above all, a technology concerning a DNA chip (or a DNA microarray) (hereinafter referred to as a biochip in this specification) is drawing attention as an effective means of gene analyses. A biochip refers to a substrate made of glass, silicon, plastics or the like with multiple different probes spotted thereon in high-density alignment. As for the probes, cDNA or short-strand nucleotides in a range from some 20- to 30-mer and the like are normally used. The elements of the biochip are based on behavior that four types of bases constituting DNA, namely, A (adenine), T (thymine), G (guanine) and C (cytosine), are coupled to each other by hydrogen bonding (i.e. A with T, and G with C); in other words, by hybridization. A target such as DNA or RNA, labeled by fluorescence materials and the like, is allowed to float on the biochip so as to hybridize with the probes, whereby the target is captured. The captured target is detected as a fluorescence signal from each spot on the biochip. By analyzing the fluorescence signals with a computer, observation of situations of several thousand to several ten thousand types of DNA or RNA in the target becomes feasible all at once.

One of applications for the biochip is sequencing by hybridization (the SBH method), which is the method used for: inspecting as to whether DNA of a target intended for investigation is contained in a sample; reading a sequence of captured DNA; or investigating polymorphic parts of DNA such as single nucleotide polymorphisms (SNPs), by means of capturing a targeted gene (or a DNA fragment).

Here, as an example, description will be made regarding bacterial identification in clinical inspection or food inspection using a. DNA of a bacterium contains the 16S ribosome RNA gene (16S rDNA) in common. Although this base sequence varies depending on each bacterium, the base sequences have clarified to date with respect to 90% of the bacteria that have been identified by 1997. Efficient use of such base-sequence information may be able to effectuate accurate determination of taxonomic positions regarding all kinds of bacteria (Hiraishi, A.: Bulletin of Japanese Society of Microbial Ecology 10, (1), 31-42, 1995).

FIG. 1 is an explanatory drawing schematically showing a method of identifying a bacterium by use of a biochip. First, base sequences in the region of 16S rDNA specific to bacteria P, Q, R and so on are selected as probes 101, 102, 103 and so on from a database 100 storing DNA sequences of bacteria, and then probe designing is performed. The respective probes corresponding to the respective bacteria are prepared in accordance with the probe designs and then the probes are spotted on a substrate as aligned lengthwise as well as sidewise, thus fabricating a biochip 104. Then, DNA extracted from blood, sputum or the like of a patient and labeled with fluorescence materials, is poured onto the biochip 104 as a target 105 so as to hybridize with the probes on the biochip 104. As a result, an assumption is herein made that signals are observed at the spot (transverse No. 1: longitudinal No. 2) as well as at the spot (transverse No. 3: longitudinal No. 5), as shown in the central part of the drawing. In this event, from a table of correspondence of spot locations to bacteria, it is understood that a bacterium [Actinobacillus actinomycetemcomitans] and a bacterium [Klebsiella oxytoca] are (possibly) mixed in the target. In this case, a signal to be detected and a bacterial strain are in a one-to-one correlation.

The conventional method of designing probes for a biochip is based on a correlation between a target and a probe on a one-to-one basis. However, such a method of designing probes has not been always satisfactory. In the first place, with the one-to-one correlation between DNA of a biological species. and a probe, there may be a case that precise judgment of the species cannot be made due to mutation or experimental errors.

Some examples of the experimental errors include: a case that a DNA fragment of a target is not coupled to the corresponding probe on a biochip with a complementary sequence to the target; or a case that a target is coupled to a probe which does not correspond to the target.

One case that the target is not coupled to the corresponding target is a case that a sequence of target DNA is different from a sequence in a public database referenced upon designing of a probe. As shown in FIG. 2, for example, if DNA of targets 202 and 203 poured onto a biochip is mutated, in other words, when single-base substitution or single-base insertion is present therein as illustrated by circles in the drawing, the targets do not hybridize with a probe 201.

Meanwhile, one case that the target is coupled to the probe not corresponding to the target is cross-hybridization. Cross-hybridization refers to a state that target genes (or DNA fragments) 302 and 304 are coupled partially to probes 301 and 303 on a biochip in the case where DNA sequences of the genes and DNA sequences of the probes are similar to each other.

According to a document (Michael D. Kane et al.: Assessment of the sensitivity and specificity of oligonucleotide (50 mer) microarrays: Nucleic Acids Res., 28(22), 4552-4557, 2000), it is reported that there is a possibility of cross-hybridization when similarity of sequences is 75% or higher, or when there are continuous complementary letter strings of 15-mer or longer even if the similarity is not relatively high (in a range from 50% to 75%).

Meanwhile, there are methods of attempting to avoid cross-hybridization, such as a method of selecting sequence-specific probes (Ken-ichi Kurata et al.: Probe Design for DNA Chips: Genome Informatics 1999, 225-6, 1999). However, those attempts are still far behind a level to avoid cross-hybridization without fail. Moreover, there is also conceived a method of predicting degrees of original fluorescence signals based on the assumption that a certain degree of cross-hybridization is present (Mitsuteru Nakao et al.: Quantitative Estimation of Cross-Hybridization in DNA Microarrays Based on a Linear Model: Genome Informatics 2000, 231-232, 2000). Nevertheless, this method has not yet reached a practical level.

Besides cross-hybridization, there are numerous possibilities that fluorescence signals are observed at spots originally not corresponding to targets, which are attributable to: experimental conditions such as temperatures during hybridization reactions and pH of a target solution; conditions of experimental instruments; or concentrations of targets and probes.

As it has been described above, whereas experimental technology concerning biochips has been improved, there still remains a possibility that an experimental error occurs. Particularly in applications of biochips to food inspection or clinical inspection, accurate identification is required. Therefore, the state of inaccurate identification as described above is undesirable. Although present biochips adopt a means for confirming repeatability of experiment by spotting multiple spots of the same probe having a certain DNA sequence onto a biochip, such means does not correspond to the experimental errors as described above.

Secondly, a biochip in which a target and a probe are correlated on the one-to-one basis cannot identify biological species on higher levels than species. A conventional chip for identifying biological species could not comply with requests for detection at a broad level, as in a case that a user intends to conduct classification not by a species of a living organism but by a genus level or a family level thereof. For example, in the case that a user intends to conduct classification of living organisms by a genus level because characteristics of the living organisms are not particularly variable at a species level, a conventional biochip cannot comply with such a demand.

Thirdly, in the event of selection of probes to be respectively specific to numerous biological species, such selection of the specific probes will reach the limit along with increases in the biological species. FIG. 4 schematically shows a state that selection of probes becomes extremely difficult upon selection of 50 probes, for example, because selected probes No. 1 to No. 50 contain DNA sequences similar to one another. Moreover, besides the similarity among the sequences, there is also a problem that Tm values among probes are not uniform when many probes are selected. A Tm value refers to a temperature at which double-strand DNA dissociates into two single strands. A hybridization reaction utilizes the behavior of DNA that double-strand DNA is dissociated into two single strands at a high temperature and the two single strands are re-formed into a double strand at a low temperature. Accordingly, a biochip requires uniform Tm values regarding probes to be spotted thereon.

### SUMMARY OF THE INVENTION

In consideration of the problems of the prior art as described above, an object of the present invention is to provide a biochip and a method of designing probes capable of detecting target genes (or DNA fragments) with higher precision and certainty. Moreover, another object of the present invention is to provide a biochip and a method of designing probes capable of identifying biological species at a broad level. Yet another object of the present invention is to provide a method of designing probes facilitating selection of species-specific probes among numerous biological species.

In order to achieve the foregoing objects, in the present invention, a plurality of different characteristic probes are designed with respect to one target. By preparing the plurality of different proves with respect to one target, identification as to which gene (or a DNA fragment) is captured becomes feasible with high certainty.

Designing probes will be conducted pursuant to the following two guidelines in accordance with objectives.

The first guideline for designing probes is selection of a plurality of partial sequences specific to target DNA from different positions on a base sequence of the target so that the partial sequences do not overlap each other. In the case of designing a plurality of probes with respect to one type of target DNA, it is undesirable that two probes specific to the sequence of the target DNA possess regions overlapping each other, because there is a risk that neither of the probes can detect the target once when the target is mutated in the overlapping position.

FIG. 5 is an explanatory drawing of a case that base sequences of two probes specific to target DNA possess regions overlapping each other. Assumption is made herein that two different probes are designed for detecting a target including a base sequence of “. . . TATCTGCGGAT . . . ”. Here, it is assumed that a sequence “ATAGACGC” complementary to an under lined part of the target “. . . TATCTGCGGAT . . . ” is selected as a first probe 501. Meanwhile, it is assumed that a sequence “GACGCCTA” complementary to an under lined part of the target “. . . TATCTGCGGAT . . . ” is'selected as a second probe 502. These two probes 501 and 502 hybridize with the target and the target can be captured at spots on a biochip where the probes 501 and 502 are fixed. However, the probes 501 and 502 possess a common region “GACGC” surrounded by frames in the drawing. For this reason, in the case that a base sequence of a target 503 to be hybridized with the probes is changed as “. . . TATCGGCGGAT . . . ” by mutation, it is likely that neither the probe 501 nor the probe 502 can capture the target because the sequences of the probes are not sequences that are completely complementary to the sequence of the mutated target.

In order to avoid such a circumstance, the plurality of probes with respect to one target should be designed so that the respective probes hybridize with regions not overlapping each other on the base sequence of the target. In this way, it is possible that any one of the probes captures the target even if mutation is occurred in the base sequence of the target, because it is extremely improbable that simultaneous mutation occurs over an entire region of the target which the probes are going to hybridize with.

FIG. 6 is a view schematically showing a mode of selecting pluralities of probes for bacteria, for example. Thin lines drawn beside Bacterium 1 to Bacterium 4 respectively show 16S rDNA (targets) of bacteria to be identified, and thick-lined portions are DNA fragments as candidates for probe designing. Regions A and A′ in FIG. 6 are the DNA fragments unique in Bacterium 1, which are regions low in homology (not similar in terms of DNA sequences) with respect to Bacterium 2, Bacterium 3 and Bacterium 4. In addition, the regions A and A′ are mutually low in homology as well. The same applies to other regions B, B′, C, and so on. In this way, responses to various experimental errors as cited in the problems in the prior art become feasible by collecting probes complementary to the regions unique and low in homology with respect to other sequences, and by preparing double or triple probes regarding each target.

The number of probes for identifying one target may vary according to purposes. For example, pursuant to degrees of importance or degrees of attention of respective bacteria upon clinical inspection, a small number of probes A and A′ may be prepared for Bacteria A of a low degree of attention and a large number of probes D, D′, D″ and so on may be prepared for Bacterium D of a high degree of attention as shown in FIG. 7. Then, bacteria of high degrees of attention can be surely detected without overlook. Moreover, in the case that detection should be focused on epidemic viruses or genetically modified novel farm products, a large number of probes should be prepared therefore. It should be noted that the probes with respect to one bacterium are disposed in alignment. However, modes of alignment of probes are not particularly limited; accordingly, such probes may be also disposed at random.

The second guideline upon designing a plurality of characteristic probes is selection of a DNA region common to some targets as a probe. For example, there may be the case that it is essential that a certain. bacterium targeted for identification is identified at a species level or at a race level but it is satisfactory that other bacteria are identified at a part level, an order level, a family level or a genus level. In the case that identification is not expected at a species level but at a broad classification level such as a part, an order, a family or a genus, it is satisfactory that a DNA sequence, which is possessed in common by bacteria of such classification, is selected as a probe. In other words, a probe unique to a family or to a genus is selected.

FIG. 8 is a view for describing selection of a probe unique to a species and selection of a probe unique to a genus. Bacteria 1, 2, 3 and 4 belong to of the genus Acinetobacter, and Bacteria 5, 6 and 7 belong to the genus Actinobacillus. In order to identify a bacterium as any one of Bacteria 1, 2, 3 and 4, i.e. as a bacterium of the genus Acinetobacter, a probe should be designed to hybridize with a portion of sequence H, which is possessed only by the bacteria of that genus in common. Similarly, in order to identifying a bacterium as a bacterium of the genus Actinobacillus, a probe should be designed to hybridize with a portion of sequence I. Actually, it is almost possible to select such common sequences. According to the International Committee on Systematic Bacteriology, one species of bacteria is defined as a group of bacteria having 70% or higher homology in quantitative DNA hybridization.

Moreover, a plurality of characteristic probes are designed in the present invention based on classification of living organisms according to a molecular dendrogram, whereby judgment as to which biological species the DNA in the target is originated from, and selection of species-specific probes among numerous biological species are facilitated. Here, the molecular dendrogram refers to a dendrogram formed on the basis of homologies in biopolymer sequences among living organisms, in which living organisms classified below one node are closely related one another and the living organism share the biologically similar nature.

The guideline for designing the plurality of characteristic probes is not to design probes in association only with one-to-one correlations of biological species as previously conducted, but it is to select a DNA sequence as a probe which is common to some targets. In this event, probe designing is conducted in response to each node by use of the molecular dendrogram as input data. That is, if there is a base sequence which is common to all bacteria below a certain node on the molecular dendrogram but not present in other bacteria, such a node is designed as a probe which is unique in that node.

FIG. 9 is a view showing an example of designing probe in line with the molecular dendrogram. Bacteria 1, 2 and 3 possess a common sequence I, and Bacteria 4 to 8 possess a common sequence L. Moreover, among the bacteria possessing the common sequence L, Bacteria 4 and 5 possess a common sequence J, and Bacteria 7 and 8 possess a common sequence K. Probes unique in Bacteria 1 to 8, respectively, are designed from sequences A, A′, B, . . . , H, H′ which are unique in the respective bacteria. Simultaneously, if there are DNA sequences common to bacteria bellow the corresponding nodes such as the sequences I, J, K and L, probes unique to those nodes are designed therefrom. When the probes corresponding to the nodes on the molecular dendrogram are designed, it is possible to recognize not only names of bacteria on detected spots but also proximity among them, whereby bacteria included in a target can be identified more precisely. As a matter of fact, whereas the molecular dendrogram is formed based on homologies in DNA sequences, it is almost coincident with an evolutionary dendrogram which is morphologically produced. For this reason, the method of classification such as species and genus, which is based on the evolutionary dendrogram, frequently coincides with relation of nodes and leaves on the molecular dendrogram. In addition, even if a probe for a unique sequence in a bacterium (a probe corresponding to a leaf in the evolutionary dendrogram) was not observed for some reason, it is still possible to place the bacterium into a position at a higher level.

In addition, the method of preparing the spots at multiple levels as shown in FIG. 9 has an advantage that the method can reduce the number of spots in comparison with the method of preparing several types of probes unique to one target. Moreover, the method of preparing the spots for at multiple levels is capable of performing more accurate judgment than simple preparation of a plurality of probes specific to bacteria, because a degree of mixture of bacteria can be synthetically discriminated by considering signals from many spots together.

Furthermore, whereas a normal probe is designed for target DNA which is clarified beforehand, multiple-level probe configuration as shown in FIG. 9 can guess a genus of a bacterium if unexpected target is contained in a sample.

Moreover, if the probes selected in accordance with FIG. 9 are disposed on a chip as shown in FIG. 10, it is feasible to check visually from fluorescence signals as to what kind of target DNA is detected. In an example shown in FIG. 10, it is possible to judge that Bacterium 1, Bacterium 3 and Bacterium 7 are mixed from probes (A, A′, C, C′, C″, G and G′). which are unique to the bacteria, as well as from probes (I, K and L) that correspond to intermediate nodes of the dendrogram. It should be noted that a similar effect is obtained by means of: arranging the probes at random on the chip instead of disposing the probes themselves on the chip as shown in FIG. 10; detecting fluorescence signals on the respective spots on the biochip; and then rearranging the fluorescence signals corresponding to the respective spots as arranged in FIG. 9 and displaying the rearranged image. on a display.

Furthermore, generally, there may be cases that sequences common to a plurality of target DNA overlap in one bacterium, such as the sequence I and the sequence J as shown in FIG. 11. By combining a plurality of probes, identification with higher reliability is effectuated.

FIG. 12 is a view showing one example of analytic result after reading fluorescence signals out of spots on a biochip. Circles in fields of the fluorescence signals correspond to spots, which show observation of stronger fluorescence as the circles become whiter. In this event, it is also possible to calculate probabilities of mixture of corresponding targets from the spots actually observed, by presetting weights (such as probabilities when errors occur and probabilities that the bacteria appear in the realm of nature) corresponding to the respective probes.

As for calculation of the probabilities, for example, there is a mode of calculation of a probability that a risk rate (a probability of erroneously judging as correct and a probability of erroneously judging as incorrect) is preset with respect to each probe, thus finding a probability of an erroneous reaction while considering the entire signal results of a plurality of probes corresponding to a certain bacterium. Assuming that a probability that a signal does not show up notwithstanding that a bacterium is actually mixed is 0.3 regarding both the probe A and the probe A′, respectively, then a probability that Bacterium 1 is mixed to a sample notwithstanding that two signals concerning the probe A and the probe A′ are weak is calculated as 0.09 (0.3×0.3). On the contrary, if a probability that a signal shows up notwithstanding that a bacterium is not actually mixed is 0.3 regarding both the probe A and the probe A′, respectively, then a probability that Bacterium 1 is not mixed to the sample when the signals concerning the probe A and the probe A′ are weak is calculated as 0.49 (0.7×0.7). Therefore, from the Bayes' theorem, it is understood that a probability that the bacterium is mixed when the two probes are weak is calculated as 0.155 (≦0.09/0.49+0.09), i.e. 15.5%.

Moreover, as shown in FIG. 13, if signals from spots K and L corresponding to intermediate nodes notwithstanding that a signal from a spot G corresponding to a species is detected, then it is conceivable that cross-hybridization is occurring at the spot G corresponding to the species. In other words, it is possible to discriminate as to whether a hybridization reaction is normally carried out by the spots corresponding to the intermediate nodes. The use of a detection method as described above effectuates more accurate detection. On the contrary, if a signal from a spot I corresponding to an intermediate node is detected notwithstanding that signals are not detected from spots A, B and C corresponding to species, it is then conceivable that DNA of an unknown species or a mutated species exists in a sample. In this case, even though identification cannot be done at a species level, identification at a higher level can be done, whereby a clue for estimating an unknown kind may be presented.

When the probes for identifying species of bacteria are selected from the 16S rDNA sequences of the respective bacteria, the respective probes should not be similar to one another. As a result, when the number of the species of bacteria is increased, selection of base sequences dissimilar to one another becomes difficult. However, as shown in FIG. 14, base sequences corresponding to the species being identical or similar to one another are still usable as probes, if they are combined with sequences corresponding to the intermediate nodes which are different from one another. In an example of FIG. 14, Bacteria 1 to 3 belong to the genus α and Bacteria 48 to 50 belong to the genus β. The probe No. 1 and the probe No. 49 have sequences closely similar to each other. Even in this case, the probes No. 1 and No. 49, which cannot be used under normal conditions because they are closely similar to each other, become usable as probes for species by simultaneous use of the probes α and β corresponding to the genera with the probes corresponding to the species. Upon detection of targets, judgments is done synthetically out of signals from a plurality of probes respectively corresponding to the species or the intermediate nodes, as described with FIG. 10 and FIG. 13.

To sum up, the characteristics of the present invention are describes as follows:

(1) A biochip having a substrate with a plurality of probes spotted thereon, in which a plurality of types of probes are spotted with respect to one target so that the probes hybridize respectively with a plurality of partial sequences specific to the target, the partial sequences not overlapping each other on a base sequence of the target.

(2) The biochip according to (1), in which a number of spots of the probes for hybridizing with a target of high attention is made more than a number of spots of the probes for hybridizing with a target of low attention.

(3) A biochip having a substrate with a plurality of probes spotted thereon, in which a probe is spotted so that the probe hybridizes specifically with a partial sequence existing in common to base sequences of a plurality of different targets.

(4) The biochip according to (3), in which the plurality of different targets are base sequences of bacteria belonging to any one of the same part, the same order, the same family and the same genus.

(5) A biochip having a substrate with a plurality of probes spotted thereon, in which a plurality of probes are spotted so that the respective probes hybridize specifically to respective targets, and a probe is spotted so that. the probe hybridizes specifically with a partial sequence existing in common to base sequences of a plurality of different targets.

(6) A biochip having a substrate with a plurality of probes spotted thereon for discriminating a plurality of types of target biopolymers, in which a probe hybridizing in common only with biopolymers below a node on a molecular dendrogram of a group of biopolymers including the plurality of types of target biopolymers is spotted as a probe corresponding to the node of the molecular dendrogram.

(7) A biochip having a substrate with a plurality of probes spotted thereon for discriminating a plurality of types of target biopolymers, in which probes hybridizing specifically with the plurality of types of target biopolymers respectively are spotted, and a probe hybridizing in common only with biopolymers below a node on a molecular dendrogram of a group of biopolymers including the plurality of types of target biopolymers is spotted as a probe corresponding to the node of the molecular dendrogram.

(8) A biochip having a substrate with a plurality of probes spotted thereon for discriminating a plurality of types of target biopolymers, in which a probe hybridizing in common only with biopolymers below a node on a molecular dendrogram of a group of biopolymers including the plurality of types of target biopolymers is spotted as a probe corresponding to the node of the molecular dendrogram, and probes hybridizing specifically with target biopolymers below the node respectively are spotted.

(9) A probe designing method, in which a plurality of probes are designed as probes to be spotted on a substrate of a biochip so that the probes hybridize respectively with a plurality of partial sequences specific to a target, the partial sequences not overlapping each other on a base sequence of the target.

(10) A probe designing method, in which a probe is designed as a probe to be spotted on a substrate of a biochip so that the probe hybridizes specifically with a partial sequence existing in common to base sequences of a group of targets composed of a plurality of different targets.

(11) A probe designing method, in which a plurality of probes are designed as probes to be spotted on a substrate of a biochip so that the probes hybridize specifically with a plurality of targets respectively, and a. probe is designed as a probe to be spotted on the substrate of the biochip so that the probe hybridizes specifically with a partial sequence existing in common to base sequences of a plurality of different targets.

(12) A probe designing method for discriminating a plurality of types of target biopolymers contained in a sample, in which a probe hybridizing in common only with biopolymers below a node on a molecular dendrogram of a group of biopolymers including the plurality of types of target biopolymers is designed as a probe corresponding to the node of the molecular dendrogram.

(13) A probe designing method for discriminating a plurality of types of target biopolymers contained in a sample, in which probes hybridizing specifically with the plurality of types of target biopolymers respectively are designed, and a probe hybridizing in common only with biopolymers below a node on a molecular dendrogram of a group of biopolymers including the plurality of types of target biopolymers is designed as a probe corresponding to the node of the molecular dendrogram.

(14) A probe designing method for discriminating a plurality of types of target biopolymers contained in a sample, in which a probe hybridizing in common only with biopolymers below a node on a molecular dendrogram of a group of biopolymers including the plurality of types of target biopolymers is designed as a probe corresponding to the node of the molecular dendrogram, and probes hybridizing specifically with target biopolymers below the node respectively are designed.

(15) A target detecting method for detecting existence of a target biopolymer based on hybridization reactions with probes, in which detection of existence of the target biopolymer is performed based on the hybridization reactions with probes including: a hybridization reaction with a probe hybridizing in common only with biopolymers below a given node on a molecular dendrogram with respect to a group of biopolymers including a plurality of types of biopolymers to be targets; and hybridization reactions with probes hybridizing specifically to the respective biopolymers below the given node.

### BRIEF DESCRIPTION OF THE DRAWINGS

FIG. 1 is an explanatory diagram schematically showing a method of identifying bacteria by use of a biochip.

FIG. 2 is an explanatory diagram of an example in which target DNA is not coupled to a probe corresponding thereto.

FIG. 3 is an explanatory diagram of an example in which target DNA is coupled to a probe which does not correspond to the target DNA.

FIG. 4 is a view describing difficulty of selection of probes in a case where numerous types of bacteria are present.

FIG. 5 is an explanatory diagram for a case that two probes specific to a sequence in a target possess regions overlapping each other.

FIG. 6 is a view showing a plurality of probes are taken from separate regions on DNA.

FIG. 7 is an explanatory diagram of designing a biochip in response to degrees of attention of target DNA.

FIG. 8 is an explanatory diagram of designing a biochip in response to information regarding species or genera of target DNA.

FIG. 9 is an explanatory diagram of designing a biochip in response to an evolutionary dendrogram generated from a set of target DNA.

FIG. 10 is a view showing an example of a biochip on which probes are disposed so as to be visually discernible with fluorescence signals as to which target DNA is emerging.

FIG. 11 is an explanatory diagram showing definition of a plurality of probes taken from common regions to a plurality of target DNA.

FIG. 12 is a view showing an analytic result after reading fluorescence signals.

FIG. 13 is a view showing another example of an experimental result using a biochip of the present invention.

FIG. 14 is a view describing that probes having identical or similar base sequences corresponding to the species are still usable as probes if base sequences corresponding to intermediate nodes are different.

FIG. 15 is a block diagram showing a configuration of a biochip system according to the present invention.

FIG. 16 is a view showing an example of a data structure of sequence data of target DNA.

FIG. 17 is a view showing an example of a data structure of sequence data of probe DNA.

FIG. 18 is a flowchart schematically showing a fabrication process of a biochip according to the present invention and a process of target detection by use of the biochip.

FIG. 19 is a flowchart showing details of determination of probe sequences.

FIG. 20 is a flowchart showing details of analysis of fluorescence signals.

FIG. 21 is a block diagram showing a configuration example of a biochip system according to the present invention.

FIG. 22 is a view showing a structure of dendrogram data.

FIG. 23 is a view showing a data structure of a node structure.

FIG. 24 is a view showing relations of linkages of a node structure.

FIG. 25 is a view showing schematic processing flow of the present invention.

FIG. 26 is a view showing detailed flow of decision of probe sequences.

FIG. 27 is a view showing an example of a display screen of results of probe selection.

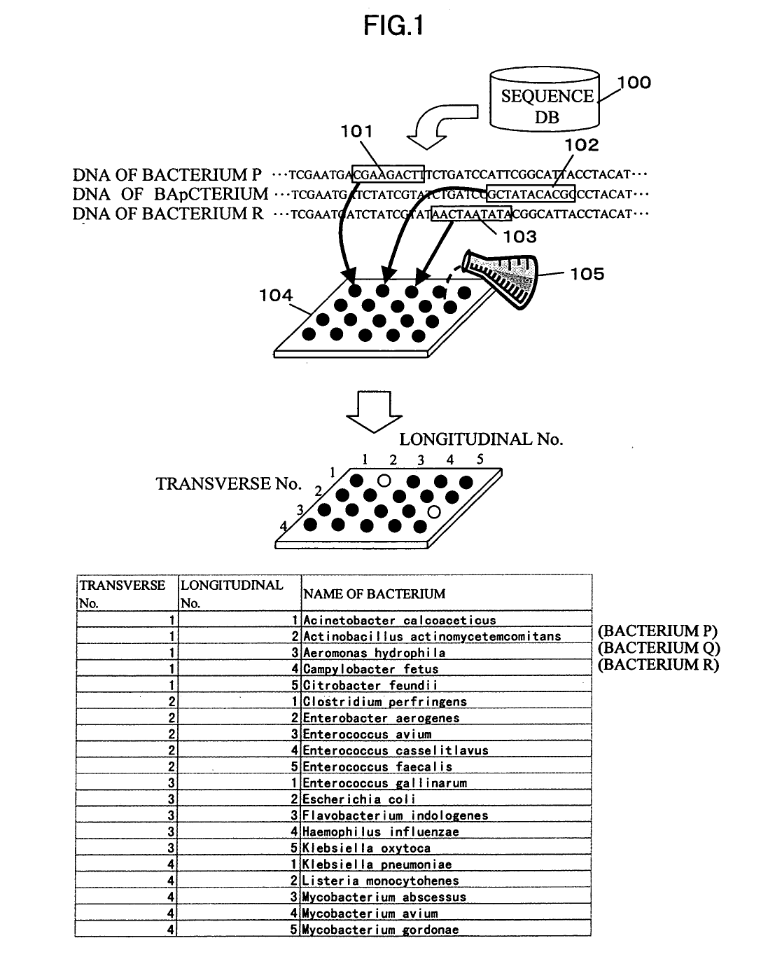

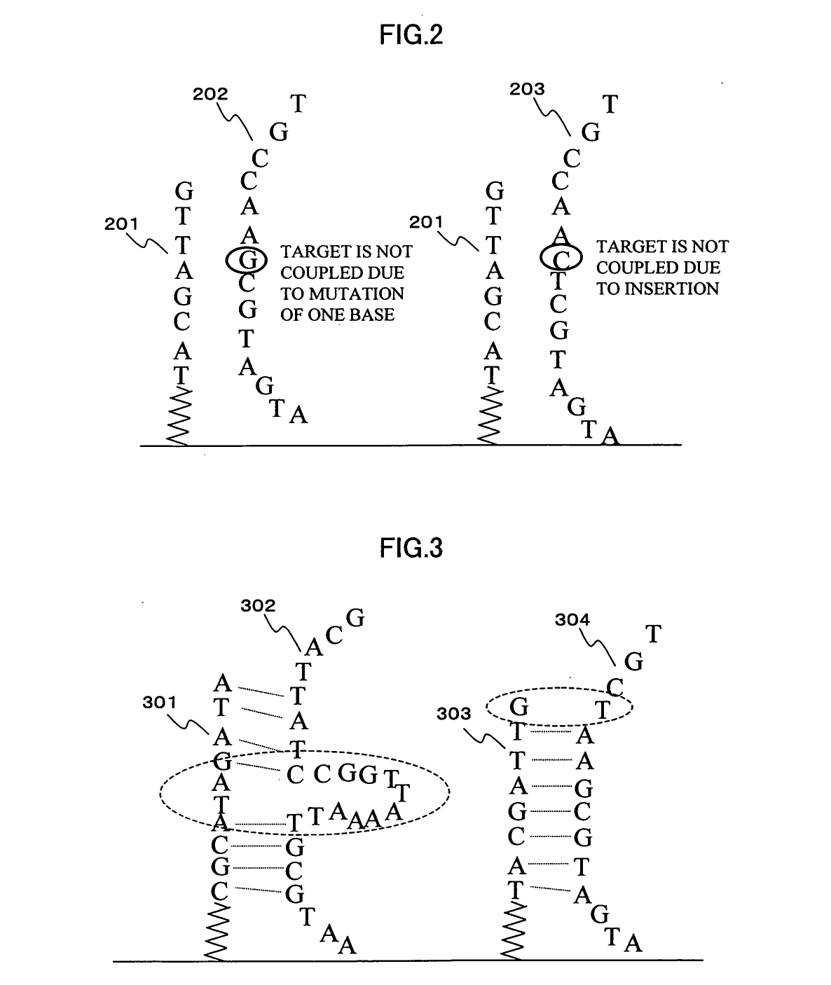

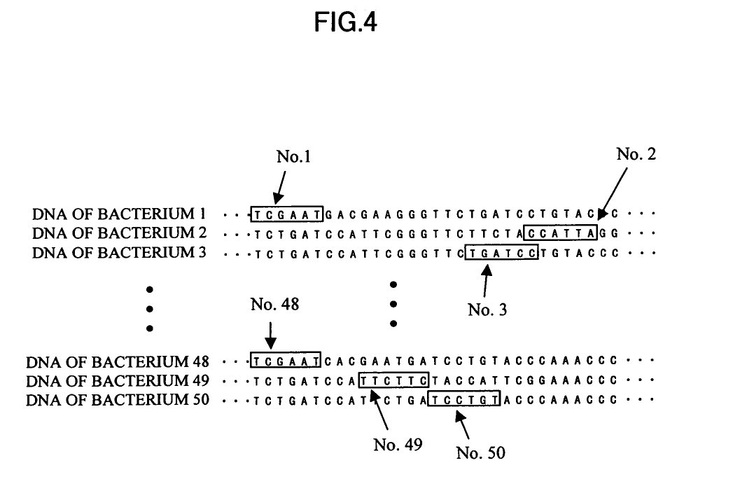

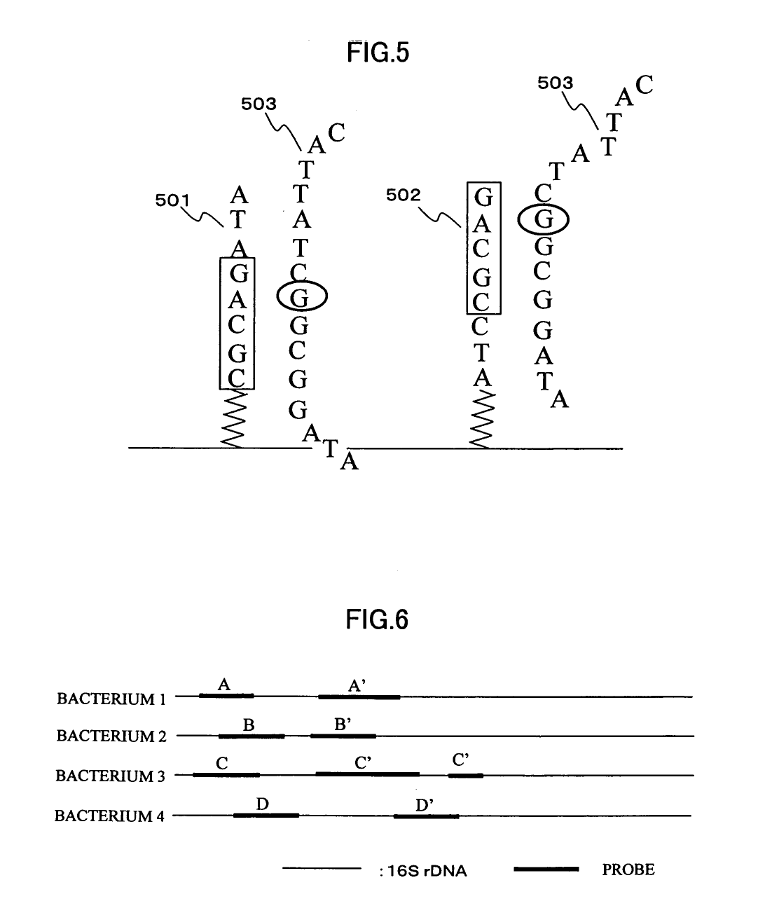

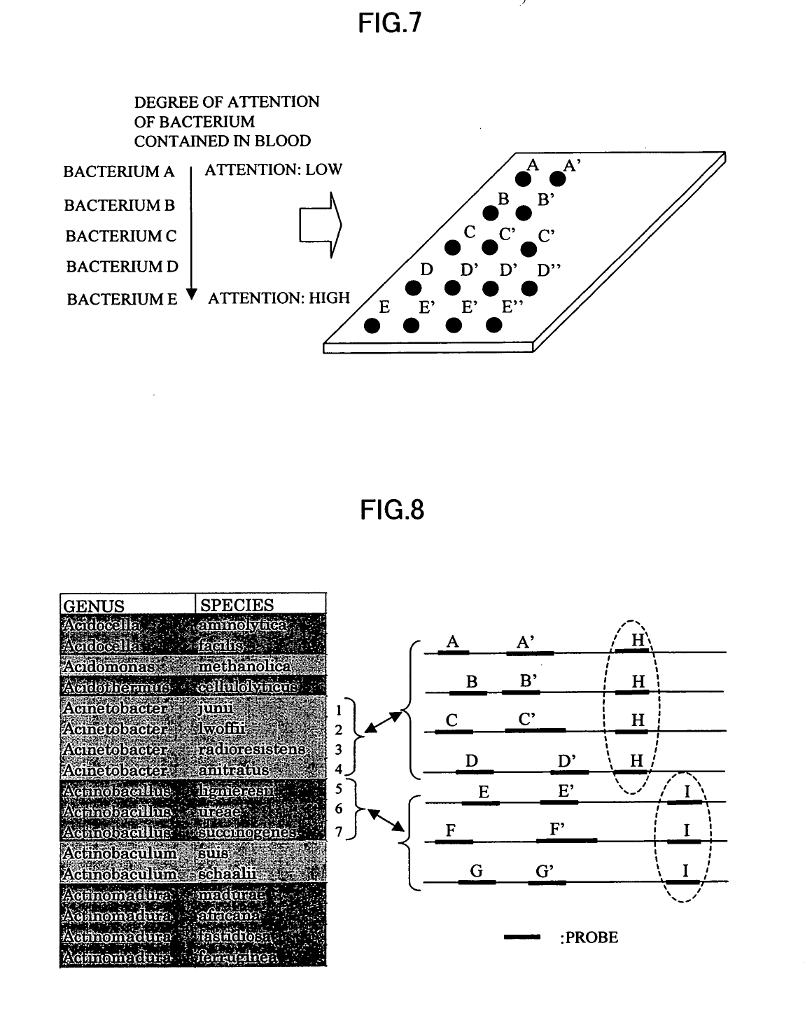

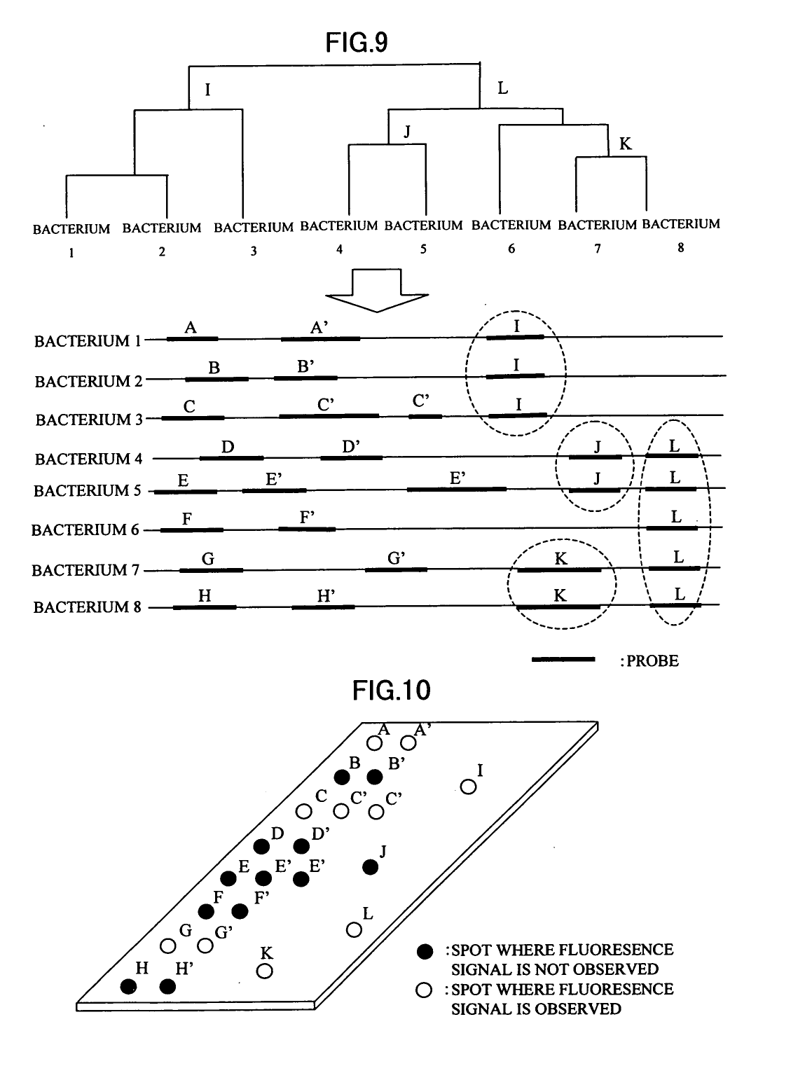

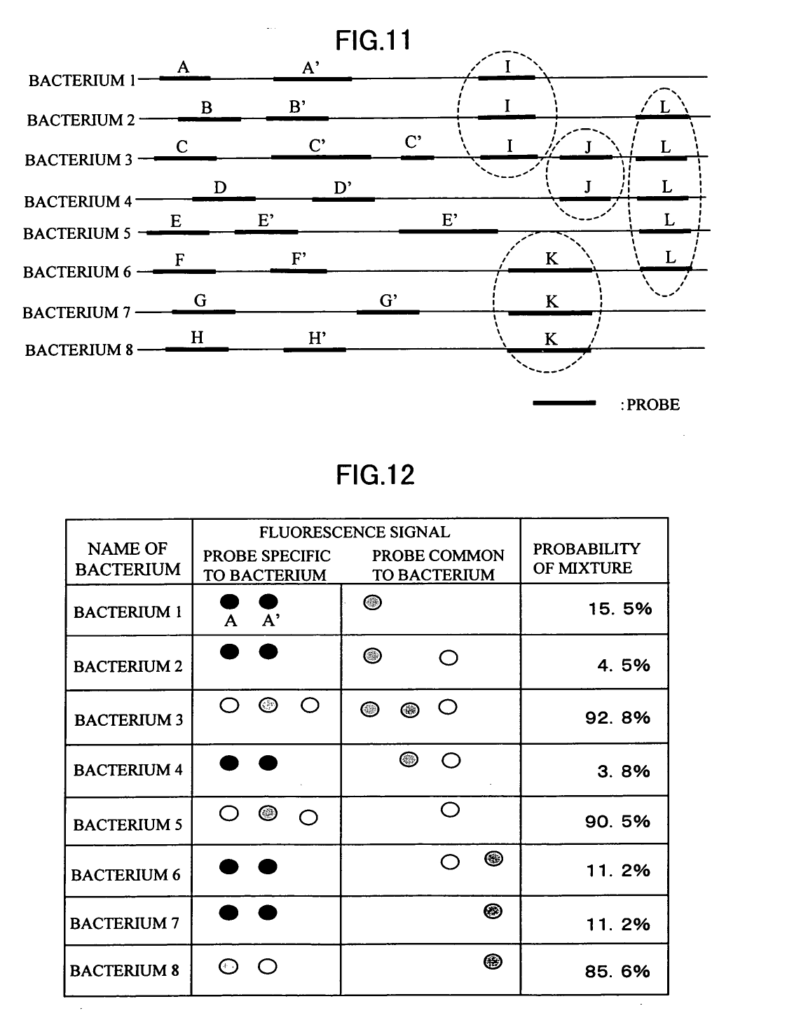

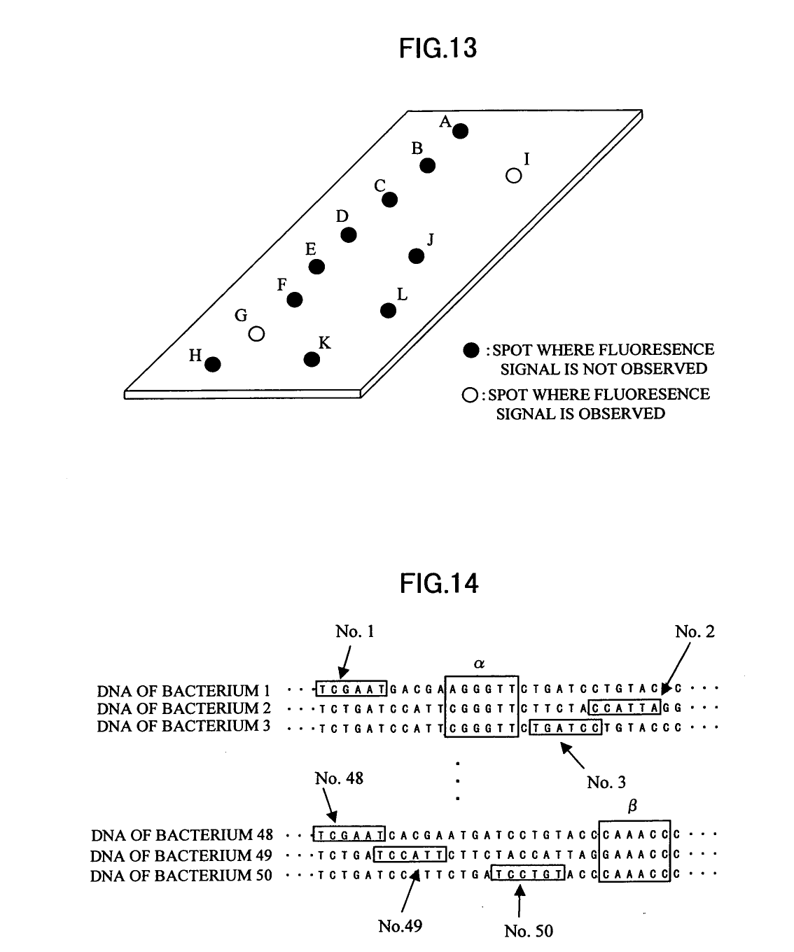

### DETAILED DESCRIPTION OF THE PREFERRED EMBODIMENT

Now, an embodiment of the present invention will be described concretely with reference to the accompanying drawings.

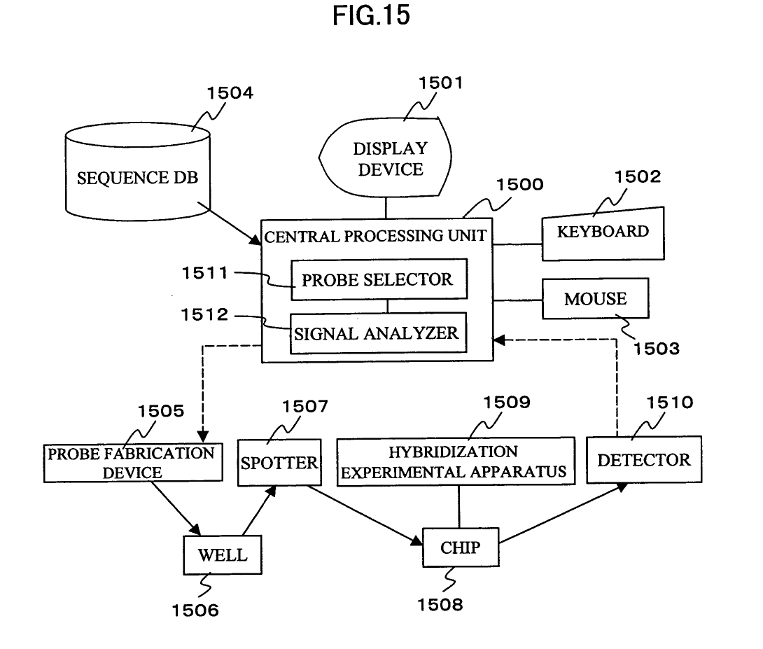

FIG. 15 is a block diagram showing an example of a configuration of a biochip system for performing fabrication of a biochip, detection of fluorescence signals and analyses of signal data.

This biochip system includes: a central processing unit 1500 for performing input/output of sequence data as well as analyses of experimental data and the like; a display device 1501 for displaying characters and graphic image screens; a keyboard 1502 and a mouse 1503 for operations to input values to the system arid to select items; and a sequence database 1504 for storing information on target DNA for use in designing probe DNA sequences. The central processing unit 1500 includes a probe selector 1511 for selecting a probe DNA sequence out of DNA sequence data and a signal analyzer 1512 for analyzing fluorescence signals read out with a detector 1510. The probe selector 1511 and the signal analyzer 1512 are materialized by a computer and programs for the computer. The sequence database 1504 may be either a local database, or a database managed by a server computer located in a remote place via a network or the like.

A probe fabrication device 1505 fabricates a probe to be mounted on an actual chip from a probe DNA sequence designed by the central processing unit 1500. The probe fabricated by the probe fabrication device 1505 is put into a well 1506, and the probe inside the well 1506 is taken out with a spotter 1507 and is spotted in a given position on a chip 1508. The probe on the biochip is subjected to hybridization with a target in a sample by a hybridization experimental apparatus 1509, and a fluorescence signal from a spot on the chip after hybridization is read out with the detector 1510. The fluorescence signal read out with the detector 1510 is then inputted to the central processing unit 1500 and is analyzed by the signal analyzer 1512.

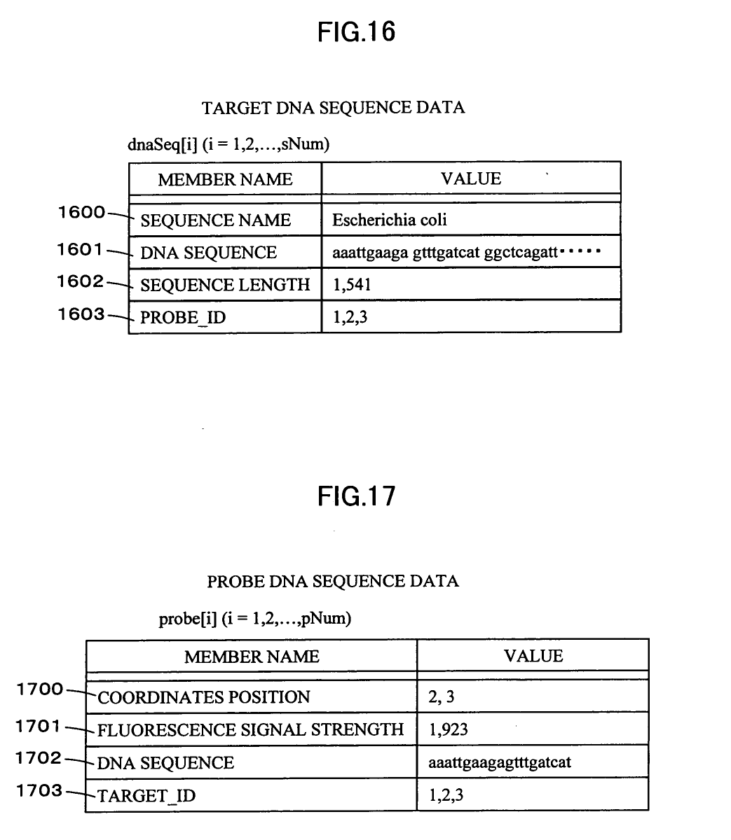

FIG. 16 is a view showing an example of sequence data of target DNA managed by the relevant system. Information on sequence data is stored into sequences dnaseq[i] (i=1, 2, . . . , sNum) having structures of elements equivalent to sNum; provided that sNum is the number of the target DNA being an object of calculation upon probe designing. A sequence dnaseq[ ] includes a sequence name (1600), a DNA sequence (1601), a sequence length of the DNA sequence (1602) and a PROBE_ID (1603) indicating which probes detect this sequence. An identifier of each probe which can identify this target DNA is inputted to the PROBE_ID. Such an identifier indicates an index of a sequence probe[ ] to be mentioned later. Moreover, in order to display attributes concerning a DNA sequence afterward, a name of an organic tissue (an organ) where the sequence is extracted from, a name of a living organism, information concerning a sequence database and the like may be also added as attributes of the dnaSeq[ ].

FIG. 17 is a view showing an example of sequence data of probe DNA managed by the relevant system. The sequence data is stored into sequences of probe[i] (i=1, 2, . . . , pNum) having structures of a length equivalent to pNum. Here, pNum is a total number of probes to be mounted on the chip. A sequence probe[ ] includes a coordinates position (1700) of a probe on the chip, a fluorescence signal intensity (1701) observed with the detector, a DNA sequence of the probe (1702) and a TARGET ID (1703) indicating a list of targets detectable by the probe. An index of the above-described sequence dnaseq[ ] is inputted to the TARGET_ID as an identifier of the target.

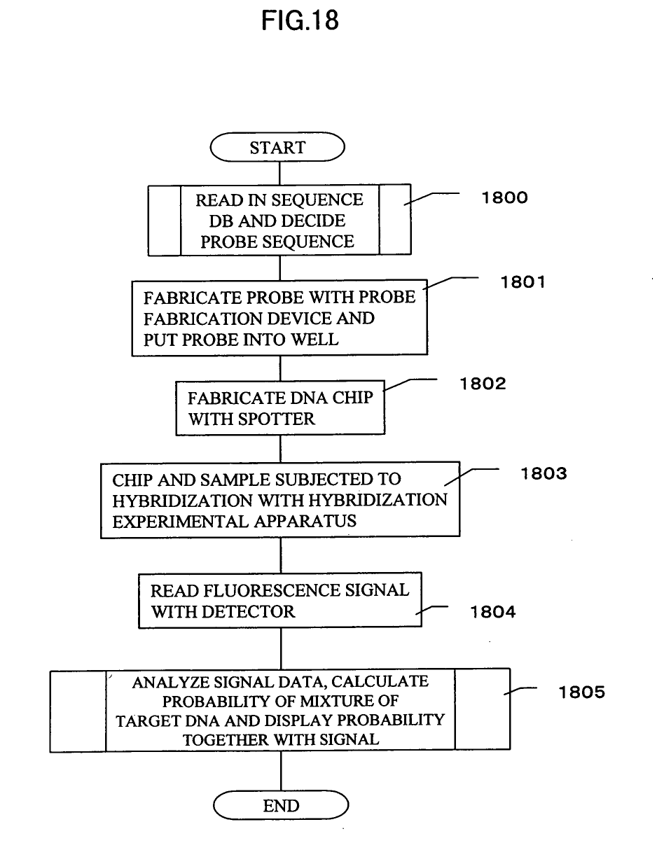

FIG. 18 is a flowchart schematically showing a fabrication process of a biochip according to the present invention and a process of target detection by use of the biochip.

First, target DNA sequence data to be objects of probe selection are read from the sequence database 1504, whereby probe DNA sequences are decided (Step 1800). The probe DNA sequences (probe[i] (i=1, 2, . . . , pNum)) decided therein are. transmitted to the probe fabrication device 1505, and probes are actually fabricated (Step 1801). The fabricated probes are put into the well 1506, and the biochip 1508 is fabricated with the spotter 1507 using the probes in the well (Step 1802). The fabricated biochip is subjected to hybridization with a sample by the hybridization experimental apparatus 1509 (Step 1803). After hybridization, fluorescence signals from the probes on the chip are read out with the detector 1510 (Step 1804). Lastly, signal data are analyzed for calculating probabilities that the target DNA exists in the sample, and then the probabilities are displayed on the display device 1501 together with signal images, to end the process (Step 1805).

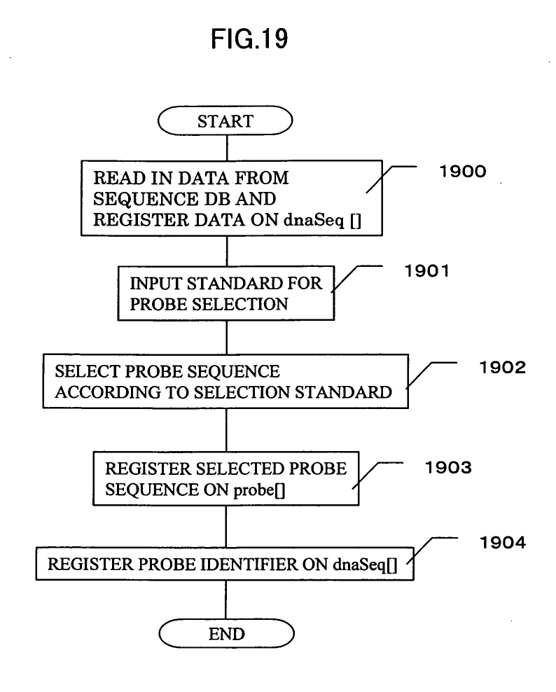

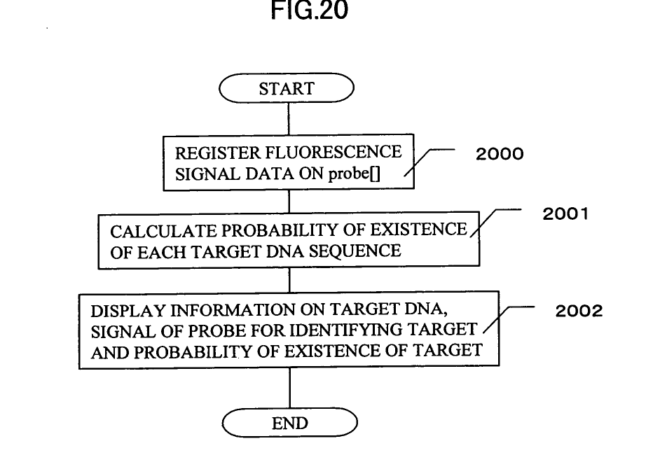

FIG. 19 shows a detailed flow of the process of deciding the probe DNA sequences by reading the sequence database (Step 1800) as described in FIG. 18.

First of all, the target DNA sequence data by sNum items to be the objects of probe designing are read from the sequence database 1504. Then, such information on target DNA sequence data is stored into the sequences dnaseq[i] (i=1, 2, . . . , sNum). In this event, names of the DNA sequences are inputted to sequence name member 1600, each of DNA sequences themselves are inputted to DNA sequence member 1601, and lengths of the DNA sequences are inputted to sequence length member 1602, respectively (Step 1900).

Next, standards for probe selection are inputted with the keyboard 1502 and the mouse 1503. In other words, information concerning requirements for selecting the probe DNA sequences are set up, such as: how many mers of probes to be fabricated; Tm values (temperatures at which double-stranded DNA is dissociated into two single strands) of the probes; and limit values of sequential similarities to other target DNA. Moreover, the following setting is concurrently carried out, concerning: how many probes unique to each target DNA should be fabricated; and which probe common to a set of target DNA should be selected. In addition to the foregoing method, methods for inputting the standards for probe selection also include a mode of reading a file in which information concerning probe fabrication is included beforehand (Step 1901).

Next, the probe DNA sequences are selected based on the standards for probe selection previously inputted. When the probes unique to the DNA sequences are selected, first a DNA partial sequence (a probe candidate) equivalent to the length of the probe is extracted from the target DNA sequence, and then the probe candidate is inspected in terms of the following points such as: whether the probe candidate is unique with respect to the entire DNA sequence; whether the probe candidate satisfies a standard Tm value; whether the probe candidate does not exceed the limit value of sequential similarities to other DNA sequences; and whether the probe candidate is not a sequence easily inducing cross-hybridization. The probe candidate which is satisfactory to these standards and the most desirable of all is selected as a probe unique to the target DNA. In the case when a plurality thereof are selected for the target DNA, the respective probe candidates should be extracted not to overlap each other, as described in FIG. 6.

Likewise, when a probe common to a plurality of target DNA is selected, a partial sequence equivalent to the length of the probe is extracted from the target DNA sequence as a probe candidate, and then the probe candidate is inspected in terms of the following points such as: whether the probe candidate is included in common to the plurality of target DNA sequences; whether the probe candidate is not included in other target DNA sequences other than the target DNA sequences; and whether the probe candidate satisfies the standards of the Tm value and the sequential similarities. Thereafter, the most desirable probe candidate is selected as a probe unique to the target DNA sequences.

In the case when any desirable probe candidate is not selected, such a fact is outputted to the display device 1501. The total number of the selected probes is referred to as pNum (Step 1902).

The probes selected in Step 1902 are stored into probe DNA sequences (probe[i] (i=1, 2, . . . , pNum)). In this event, the probe DNA sequences are inputted to DNA sequence member 1702, and the index of the dnaseq[ ] corresponding to the target detectable with the probes are inputted to TARGET_ID member 1703 of the probe[ ], respectively. In addition, coordinates of the probes disposed on the biochip are inputted to coordinates position member 1700 of the probe[ ]. As shown in FIG. 10, a mode of usage is conceivable therein to dispose the coordinates of the probes into a formation so that mixture of the target is visually discernible. The foregoing operation is performed with respect to all pNum items of the probes selected in Step 1902 (Step 1903).

Next, the identifiers of the probes are inputted to the PROBE_ID member of the dnaSeq[ ]. In other words, when an index “j” is registered as a value for the TAGET_ID member of probe[i], then “i” is inputted to the PROBE_ID member of the dnaSeq[j] (Step 1904). Now, the process is completed.

FIG. 20 shows detailed flow of the process of calculating the probabilities of mixture of the target DNA by analyzing the signal data, and displaying the probabilities together with the signals (Step 1805) as described in FIG. 18.

First of all, the signal data read out with the detector 1510 in Step 1804 are stored into fluorescence signal intensity member 1701 of the probe[ ] (Step 2000). Then, the probabilities of existence of the respective target DNA sequences are calculated according to the signal data. As for a method of calculating the probabilities, for example, the signals of the respective DNA sequences are substituted with 1 when intensities thereof are strong and 0 when intensities thereof are weak, by setting a proper threshold. In addition, a risk rate (a probability pᵢ of judging erroneously that the signal is present notwithstanding that the signal is not supposed to be present, and a probability p′ᵢ of judging erroneously that the signal is not present notwithstanding that the signal is actually present) is preset with respect to each probe. In this way, regarding a certain DNA sequence, for example, when there are three probes unique-to the DNA sequence and signals are observed with respect to all those probes, then a probability of mixture of the DNA sequence can be found in accordance with the Bayes' theorem as (1−p′₁)(1−p′₂)(1−p′₃)/(p₁p₂p₃+(1p′₁)(1−p′₂)(1−p′₃)) (Step 2001).

Next, the information on the respective target DNA, signals of the probes discriminating the targets, and the probabilities of existence of the targets are displayed collectively on the display device 1501. In other words, the sequence names 1600 of the dnaseq[i] and the sequence lengths 1602 with respect to i=1, . . . , sNum are displayed as the information on the respective target DNA. Moreover, the indices registered on the PROBE_ID 1603 are traced to the probes[ ], whereby the images of the fluorescence signals are obtained from the coordinate positions 1700 of the probes[ ] and are displayed as the signals of the probes discriminating the targets. Furthermore, the probabilities calculated in Step 2001 are displayed as the probabilities of existence of the targets, whereby the process is completed (Step 2002).

In accordance with the process as described above, it is feasible to conduct proper selection of the probes, to be the objects of discrimination of the target DNA which a user intends to investigate.

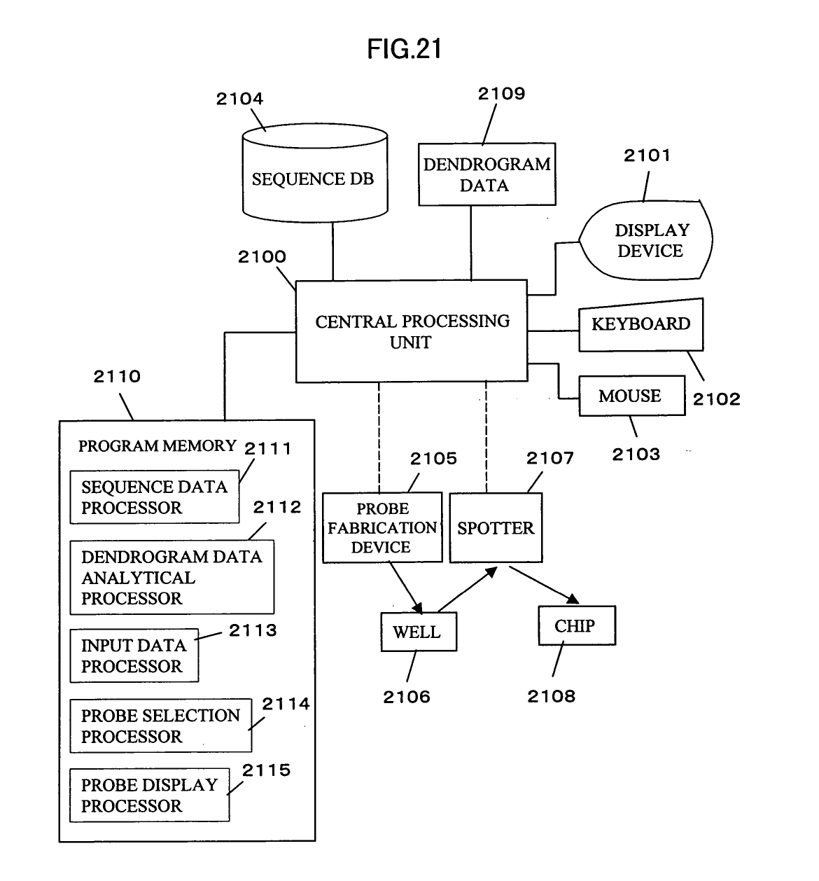

FIG. 21 is a block diagram showing another configuration example of a biochip system for performing fabrication of a biochip, detection of fluorescence signals and analyses of signal data. This biochip system includes: a central processing unit 2100 for performing input/output of sequence data as well as analyses of experimental data and the like; a program memory 2110 for storing programs required for processing at the central processing unit 2110; a display device 2101 for displaying characters and graphic image screens; a keyboard 2102 and a mouse 2103 for operations to input values to the system and to select items; a sequence database 2104 for storing information on target DNA for use in designing of probe DNA sequences; and dendrogram data 2109 that stores information on a dendrogram for use in designing -node probes.

Here, the sequence database 2104 may be either a local database, or a database managed by a server computer located in a remote place via a network or the like. The dendrogram data 2109 may be either previously-created data, or data newly created from the sequence database 2104. Moreover, the dendrogram data may be either data residing in a local computer, or data managed by a server computer located in a remote. place via a network or the like. The central processing unit 2100 is materialized by a computer and programs for the computer.

The program memory 2110 includes: a sequence data processor 2111 for processing data in the sequence database 2104; a dendrogram data analytic processor 2112 for analyzing the dendrogram data 2109; an input data processor 2113 for processing input from the keyboard 2102 and the mouse 2103; a probe selection processor 2114 for performing selective processing of probes based on a processing result by the sequence data processor 2111 as well as based on an analytic result by the dendrogram data analytic processor 2112, and a probe display processor 2115 for displaying designed probes.

The central processing unit 2100 also performs control of a probe fabrication device 2105 for fabricating a probe to be mounted on an actual chip from a designed probe DNA sequence, and performs control of a spotter 2107, which takes the probe out of a well 2106 for putting the probe therein which is fabricated by the probe fabrication device and loads the probe onto a given position on a chip 2108.

The target DNA sequence data managed by the relevant system are similar to those described with reference to FIG. 16 in the previous example, and the probe DNA sequence data herein are similar to those described with reference to FIG. 17 in the previous example.

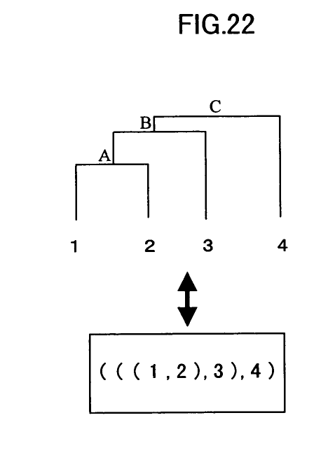

FIG. 22 shows an example of the dendrogram data, which are the data inputted to this system. The dendrogram data are formed in a file format, in which leaves of the dendrogram correspond to the identifier of the dnaSeq[ ], and a pair of parentheses correspond to one intermediate node. Moreover, when an intermediate node includes another intermediate node (which is closer to a leaf on the dendrogram), such relations are expressed with a nested structure. That is, according to the Backus Naur Form (BNF), the dendrogram data are expressed as:

node::=(node, node)|dnaSeq[ ] identifier.

Moreover, nodes corresponding to this route are written in the dendrogram data. In the example of the dendrogram data as described in FIG. 22, (1, 2) corresponds to Node A and ((1, 2), 3) corresponds to Node B.

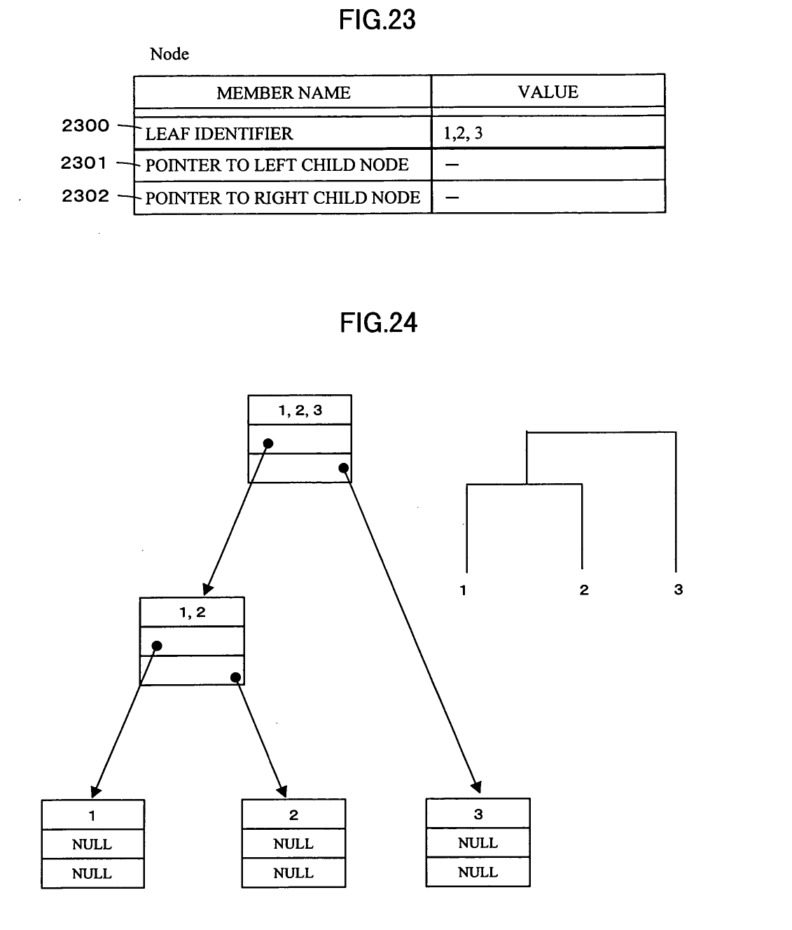

FIG. 23 is a view showing a node structure which is managed by this system. The node structure refers to a representation of each node and relevant leaves on a dendrogram. A node is composed of a leaf identifier 2300, a pointer 2301 to a left child node, and a pointer 2302 for a right child node. When a node is an intermediate node on a dendrogram, an identifier of leaves (the index of the dnaSeq[ ]) subordinate to the node is registered on the leaf identifier 2300. When the node itself is a leaf, then the index of the corresponding dnaseq[ ] is registered on the leaf identifier 2300. Moreover, when the node is the leaf, the pointer to a left node and the pointer to a right child node are filled with NULL.

FIG. 24 shows relations among the node structures, in which a tree structure of a dendrogram is reproduced by bonding the pointers to left child nodes and the pointers to right nodes together.

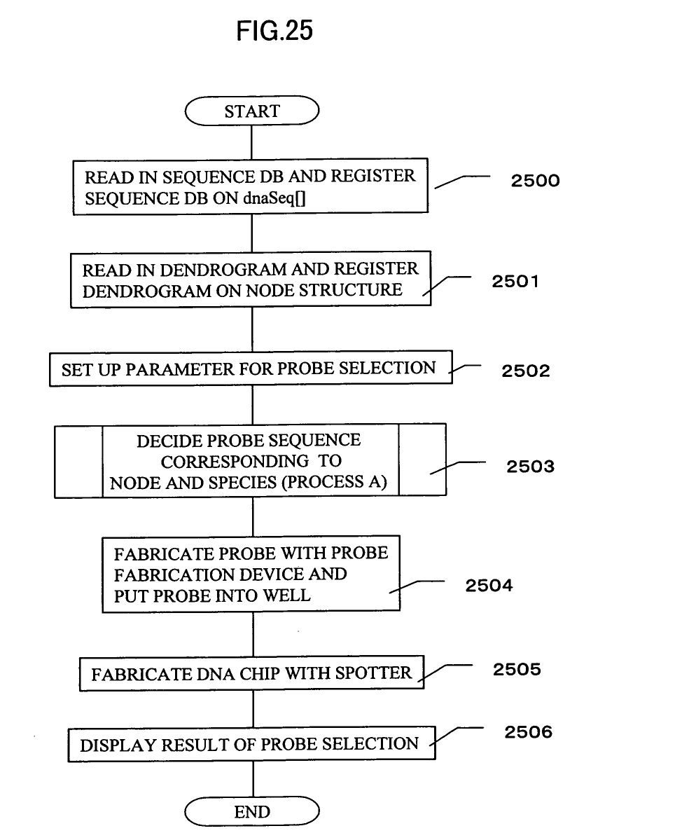

FIG. 25 is a view showing schematic processing flow of the present invention. First, target DNA data to be the objects of probe selection are read out from the sequence database 2104 and are registered on the dnaseq[ ] (Step 2500). Next, the dendrogram data are read out from the dendrogram data 2109 and are registered on the node structure. The dendrogram data 2109 may be either previously-created data, or data newly created from the sequence database. The inputted dendrogram data start building links of node structures in conformity to a formation of the dendrogram as shown in FIG. 24 (Step 2501).

Next, standards for probe selection are inputted with the keyboard 2102 and the mouse 2103. In other words, information concerning requirements for selecting the probe DNA sequences are set up, such as: how many mer of probes to be fabricated; Tm values (temperatures at which double-stranded DNA is dissociated into two single strands) of the probes; and limit values of sequential similarities to other target DNA. In addition to the foregoing method, methods for inputting the strands also include a mode of reading a file in which information concerning probe fabrication is included beforehand (Step 2502). Thereafter, by utilizing the dnaseq[ ] and the nodes, probe DNA sequences corresponding to the nodes on the dendrogram and to species are decided (Step 2503). This process will be described later in detail. Probes are stored into sequences probe[i] (i=1, 2, . . . , pNum) in accordance with this process.

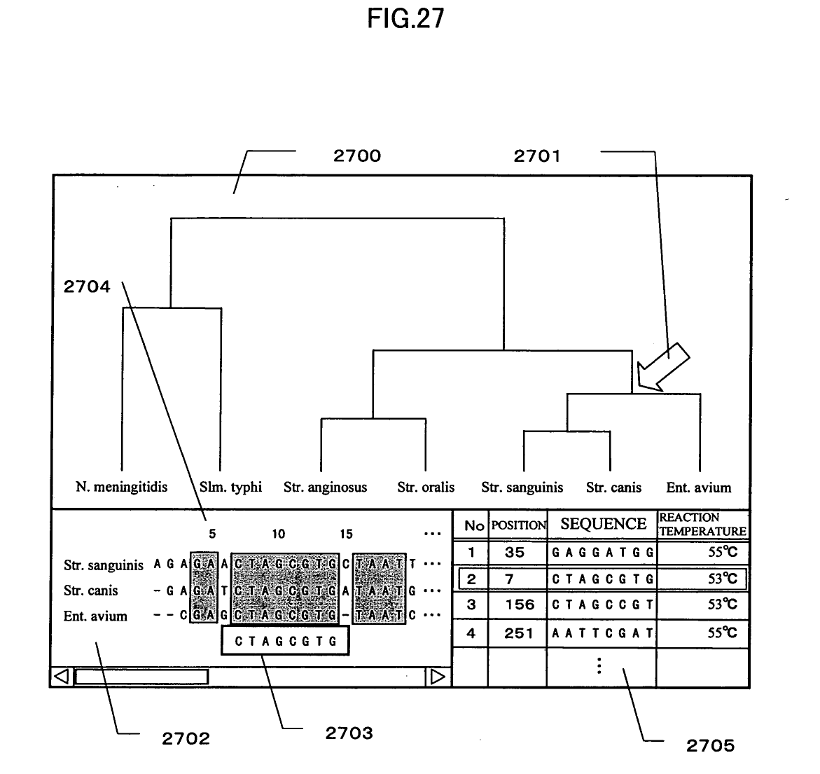

The sequences are then transmitted to the probe fabrication device 2105, whereby the probes are actually fabricated (Step 2504). The fabricated probes are coordinated into the well 2106, and then a biochip is fabricated with the spotter 2107 using the probes in the well (Step 2505). Lastly, results of probe selection corresponding to the dendrogram are displayed on the display device as shown in FIG. 27. Description will be made in detail regarding FIG. 27 later.

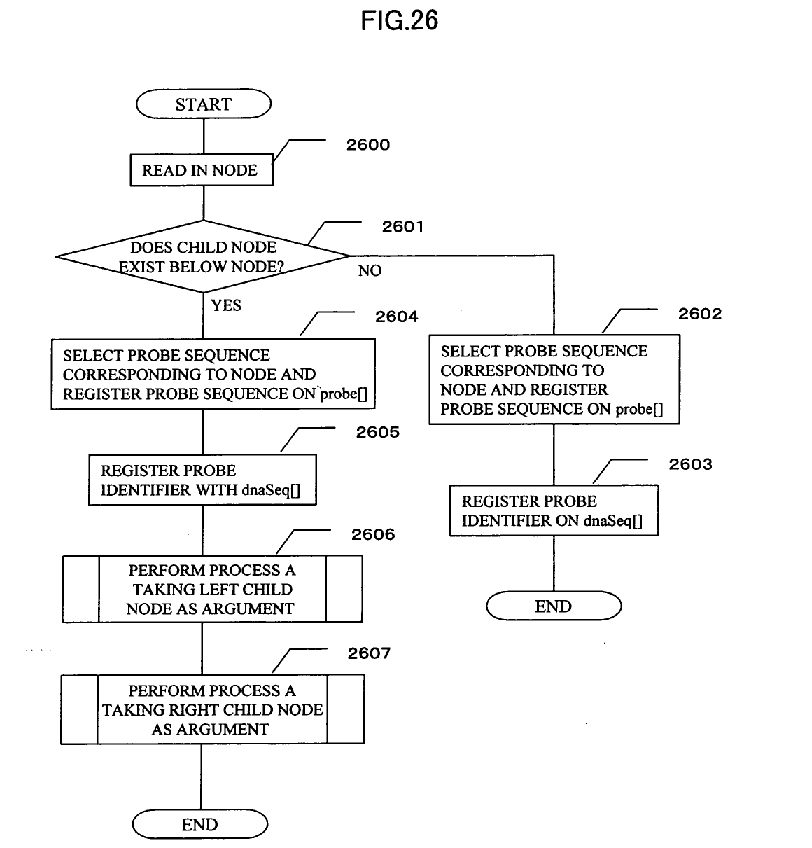

FIG. 26 shows a detailed flow regarding the process of deciding the probe DNA sequences (Step 2503) according to FIG. 25. In Step 2503 of FIG. 25, routes of the dendrogram are given to the process as arguments and the process is called.

In FIG. 26, node structure data given as arguments are firstly read in (Step 2600). Next, existence of child nodes below this node is investigated (Step 2601). If no child nodes exist, then the node corresponds to a species on a dendrogram. If a child node exists, then the node corresponds to a node on the dendrogram.

When any child nodes do not exist below the node, then a probe DNA sequence with respect to a target corresponding to the leaf identifier member 2300 of this node is selected to begin with. Then, a DNA partial sequence (a probe candidate) equivalent to a length of a probe is taken out of the target DNA sequence. Thereafter, the probe candidate is inspected in terms of the following points such as: whether the probe candidate is unique with respect to the entire DNA sequence; whether the probe candidate satisfies a standard Tm value; whether the probe candidate does not exceed the limit value of sequential similarities to other DNA sequences; and whether the probe candidate is not a sequence easily inducing cross-hybridization. The probe candidate which is satisfactory to these standards and the most desirable of all is selected as a probe unique to the target DNA. Now, the selected probe DNA sequence is registered on the DNA sequence 1702 of the probe[ ], and the leaf identifier member of the node is added to the TARGET_ID 1703 (Step 2602). The identifier for the selected probe is added to the PROBE_ID member 1603 of the dnaseq[ ] corresponding to the leaf identifier member of the node (Step 2603).

When a child node exists below the node in Step 2601, then a probe DNA sequence corresponding to this node is selected to begin with. The probe corresponding to the node must be the probe which reacts to all the species below the node but does not react with any other species. Accordingly, a partial sequence equivalent to a length of a probe is sought as a probe candidate, such that the partial sequence is included in target DNA sequences of the identifiers indicated in the leaf identifier member of the node but the partial sequence is not included in any other target DNA sequences. Thereafter, the probe candidate is inspected as to whether the probe candidate satisfies the standards of the Tm value and the sequential similarities, and the most desirable probe candidate is selected as a probe unique to the DNA sequences. The selected probe DNA sequence is registered on the DNA sequence 1702 of the probe[ ], and the leaf identifier member of the node is added to the TARGET ID 1703 (Step 2604). The identifier for the selected probe is added to the PROBE_ID member 1603 of the dnaseq[ ] corresponding to the leaf identifier member of the node (Step 2605).

Subsequently, the process from Step 2600 and thereafter is iterated regarding the left and the right child nodes of the node taken as an argument, respectively (Steps 2606 and 2607). In this way, probes are selected while circulating all the nodes and the species on the dendrogram. Moreover, if a desirable probe candidate is not obtained, such a result is outputted to the display device 2101.

FIG. 27 is a view showing an example of a screen of the display device 2101 displaying information on the probes selected by this system. When the dendrogram data 2109 are read in and displayed on a display screen 2700, a node on the dendrogram is selected by use of a cursor 2701 of the mouse 2103. Aside from the mouse 2103, selection of a node may also be carried out with the keyboard 2102. Then, reference numerals 2702, 2703, 2704 and 2705 are displayed. The reference numeral 2702 shows results of multiple alignments regarding biological species (which are 3 species of Str. sanguini, Str. Canis and Ent. avium therein) which belong to the node selected with the mouse cursor 2701. Halftone portions refer to parts of DNA sequences coincident among those 3 biological species. Non-halftone portions refer to parts of the DNA sequences which do not coincide with respect to one biological species at least. The reference numeral 2703 shows one of the probes corresponding to the node selected with the cursor 2701. The reference numeral 2704 indicates locations of the probe in the DNA sequences. Since the sequence 2703 starts from the seventh base, it is displayed from the seventh base on the multiple alignments. The reference numeral 2705 is a table of the probes corresponding to the node selected with the cursor 2701. Although probe numbers, sequences, positions in the DNA sequences and reaction temperatures are displayed therein, information such as degrees of self-interlacement of the probes or other conditions may also be displayed.

In accordance with the process as described above, it is feasible to conduct proper selection of probes to be the objects of discernment of a biological species which target DNA to be investigated is originated from.

According to the present invention, it is feasible to obtain a biochip capable of detecting a target gene (or a DNA fragment) with precision or at a desired classification level. Moreover, selection of probes can be readily performed if types of targeted DNA intended for investigation are increased. Since each of the probes corresponds to a relation of nodes and leaves on a dendrogram, the probes also play roles for error checks upon hybridization reactions or upon signal reading.

1. (Cancelled)

2. A biochip having a substrate with a plurality of probes spotted thereon, wherein a plurality of types of probes are spotted with respect to one target so that the probes hybridize respectively with a plurality of partial sequences specific to the target, the partial sequences not overlapping each other on a base sequence of the target, wherein a number of spots of the probes for hybridizing with a target of high attention is made more than a number of spots of the probes for hybridizing with a target of low attention.

3. A biochip having a substrate with a plurality of probes spotted thereon, wherein a probe is spotted so that the probe hybridizes specifically with a partial sequence existing in common to base sequences of a plurality of different targets.

4. The biochip according to claim 3, wherein the plurality of different targets are base sequences of bacteria belonging to any one of the same part, the same order, the same family and the same genus.

5. A biochip having a substrate with a plurality of probes spotted thereon,
wherein a plurality of probes are spotted to so that the respective probes hybridize specifically to respective targets, and
a probe is spotted so that the probe hybridizes specifically with a partial sequence existing in common to base sequences of a plurality of different targets.

6. A biochip having a substrate with a plurality of probes spotted thereon for discriminating a plurality of types of target biopolymers, wherein a probe hybridizing in common only with biopolymers below a node on a molecular dendrogram of a group of biopolymers including the plurality of types of target biopolymers is spotted as a probe corresponding to the node of the molecular dendrogram.

7. A biochip having a substrate with a plurality of probes spotted thereon for discriminating a plurality of types of target biopolymers,
wherein probes hybridizing specifically with the plurality of types of target biopolymers respectively are spotted, and
a probe hybridizing in common only with biopolymers below a node on a molecular dendrogram of a group of biopolymers including the plurality of types of target biopolymers is spotted as a probe corresponding to the node of the molecular dendrogram.

8. A biochip having a substrate with a plurality of probes spotted thereon for discriminating a plurality of types of target biopolymers,
wherein a probe hybridizing in common only with biopolymers below a node on a molecular dendrogram of a group of biopolymers including the plurality of types of target biopolymers is spotted as a probe corresponding to the node of the molecular dendrogram, and
probes hybridizing specifically with target biopolymers below the node respectively are spotted.

9. A probe designing method, wherein a plurality of probes are designed as probes to be spotted on a substrate of a biochip so that the probes hybridizing respectively with a plurality of partial sequences specific to a target, the partial sequences not overlapping each other on a base sequence of the target.

10. A probe designing method, wherein a probe is designed as a probe to be spotted on a substrate of a biochip so that the probe hybridizes specifically with a partial sequence existing in common to base sequences of a group of targets composed of a plurality of different targets.

11. A probe designing method,
wherein a plurality of probes are designed as probes to be spotted on a substrate of a biochip so that the probes hybridize specifically with a plurality of targets respectively, and
a probe is designed as a probe to be spotted on the substrate of the biochip so that the probe hybridizes specifically with a partial sequence existing in common to base sequences of a plurality of different targets.

12. A probe designing method for discriminating a plurality of types of target biopolymers contained in a sample, wherein a probe hybridizing in common only with biopolymers below a node on a molecular dendrogram of a group of biopolymers including the plurality of types of target biopolymers is designed as a probe corresponding to the node of the molecular dendrogram.

13. A probe designing method for discriminating a plurality of types of target biopolymers contained in a sample,
wherein probes hybridizing specifically with the plurality of types of target biopolymers respectively are designed, and
a probe hybridizing in common only with biopolymers below a node on a molecular dendrogram of a group of biopolymers including the plurality of types of target biopolymers is designed as a probe corresponding to the node of the molecular dendrogram.

14. A probe designing method for discriminating a plurality of types of target biopolymers contained in a sample,
wherein a probe hybridizing in common only with biopolymers below a node on a molecular dendrogram of a group of biopolymers including the plurality of types of target biopolymers is designed as a probe corresponding to the node of the molecular dendrogram, and
probes hybridizing specifically with target biopolymers below the node respectively are designed.

15. A target detecting method for detecting existence of a target biopolymer based on hybridization reactions with probes, wherein detection of existence of the target biopolymer is performed based on the hybridization reactions with probes including:
a hybridization reaction with a probe hybridizing in common only with biopolymers below a given node on a molecular dendrogram with respect to a group of biopolymers including a plurality of types of biopolymers to be targets; and
hybridization reactions with probes hybridizing specifically with the respective biopolymers below the given node.


SEQUENCE LISTING (22 sequences)

SEQ ID NO: 1 - DNA, 11 bp, Artificial Sequence
  Description of Artificial Sequence Target DNA
tatctgcggat

SEQ ID NO: 2 - DNA, 11 bp, Artificial Sequence
  Description of Artificial Sequence Target DNA
tatcggcggat

SEQ ID NO: 3 - DNA, 43 bp, Artificial Sequence
  Description of Artificial Sequence Synthetic probe
tcgaatgacgaagactttctgatccattcggcattacctacat

SEQ ID NO: 4 - DNA, 43 bp, Artificial Sequence
  Description of Artificial Sequence Synthetic probe
tcgaatgatctatcgtatctgatccgctatacacgccctacat

SEQ ID NO: 5 - DNA, 43 bp, Artificial Sequence
  Description of Artificial Sequence Synthetic probe
tcgaatgatctatcgtataactaatatacggcattacctacat

SEQ ID NO: 6 - DNA, 14 bp, Artificial Sequence
  Description of Artificial Sequence Target DNA
tgccaagcgtagta

SEQ ID NO: 7 - DNA, 15 bp, Artificial Sequence
  Description of Artificial Sequence Target DNA
tgccaactcgtagta

SEQ ID NO: 8 - DNA, 10 bp, Artificial Sequence
  Description of Artificial Sequence Synthetic probe
atagatacgc

SEQ ID NO: 9 - DNA, 25 bp, Artificial Sequence
  Description of Artificial Sequence Target DNA
gcattatccggttaaaattgcgtaa

SEQ ID NO: 10 - DNA, 14 bp, Artificial Sequence
  Description of Artificial Sequence Target DNA
tgctaagcgtagta

SEQ ID NO: 11 - DNA, 31 bp, Artificial Sequence
  Description of Artificial Sequence Synthetic probe
tcgaatgacgaagggttctgatcctgtacnc

SEQ ID NO: 12 - DNA, 31 bp, Artificial Sequence
  Description of Artificial Sequence Synthetic probe
tctgatccattcgggttcttctaccattagg

SEQ ID NO: 13 - DNA, 31 bp, Artificial Sequence
  Description of Artificial Sequence Synthetic probe
tctgatccattcgggttctgatcctgtaccc

SEQ ID NO: 14 - DNA, 31 bp, Artificial Sequence
  Description of Artificial Sequence Synthetic probe
tcgaatcacgaatgatcctgtacccaaaccc

SEQ ID NO: 15 - DNA, 31 bp, Artificial Sequence
  Description of Artificial Sequence Synthetic probe
tctgatccattcttctaccattcggaaaccc

SEQ ID NO: 16 - DNA, 31 bp, Artificial Sequence
  Description of Artificial Sequence Synthetic probe
tctgatccatnctgatcctgtacccaaaccc

SEQ ID NO: 17 - DNA, 15 bp, Artificial Sequence
  Description of Artificial Sequence Target DNA
cattatcggcggata

SEQ ID NO: 18 - DNA, 30 bp, Artificial Sequence
  Description of Artificial Sequence Target DNA
aaattgaagagtttgatcatggctcagatt

SEQ ID NO: 19 - DNA, 20 bp, Artificial Sequence
  Description of Artificial Sequence Target DNA
aaattgaagagtttgatcat

SEQ ID NO: 20 - DNA, 20 bp, Streptococcus sanguinis
agagaactagcgtgctaatt

SEQ ID NO: 21 - DNA, 19 bp, Streptococcus canis
gagatctagcgtgataatg

SEQ ID NO: 22 - DNA, 17 bp, Enterococcus avium
cgagctagcgtgtaatc